(19) United States
(12) Patent Application Publication
(10) Pub. No.: US 2004/0247596 A1
(43) Pub. Date: Dec. 9, 2004

(54) TRANCE regulation of chondrocyte differentiation

(76) Inventors: Paul R. Odgren, Princeton, MA (US); Sandy C. Marks JR., Westboro, MA (US); Yongwon Choi, Bryn Mawr, PA (US)

Correspondence Address: J. PETER FASSE, Fish & Richardson P.C., 225 Franklin Street, Boston, MA, 02110-2804, US

(21) Appl. No.: 09/933,915

(22) Filed: Aug. 20, 2001

Related U.S. Application Data
(60) Provisional application No. 60/226,197, filed on Aug. 18, 2000.

(51) Int. Cl. A61K 39/395; A61K 48/00

(52) U.S. Cl. 424/145.1; 514/44

(57) ABSTRACT

Disclosed are therapeutic methods of treating a mammal, e.g., a human patient, having a disease, disorder or condition characterized by abnormal (excessive or insufficient) cartilage growth or skeletal growth. The methods include inhibiting or supplementing activity of TRANCE or TRAF6 in chondrocytes in vivo or ex vivo. Also disclosed are methods of diagnosing a cartilage disorder. The method includes detecting an elevated or reduced level of TRANCE, RANK, or TRAF6 in chondrocytes. Also disclosed are methods of identifying a compound that increases or decreases proliferation of chondrocytes, or a compound that promotes differentiation, e.g., maturation, of chondrocytes.

### CROSS REFERENCE TO RELATED APPLICATION

This application claims priority from U.S. Provisional Application Ser. No. 60/226,197 filed on Aug. 18, 2000, which is incorporated herein by reference in its entirety.

### FEDERALLY SPONSORED RESEARCH

Work on this invention was supported in part by NIH (NIDCR) Grant No. DE 07444. Therefore, the government has certain rights in the invention.

### TECHNICAL FIELD

This invention relates to molecular biology, cell biology, anatomy and medicine.

### BACKGROUND

The skeleton is a dynamic system throughout life, balancing the need for physical support and locomotion with the requirement for precisely regulated concentrations of circulating mineral ions. These processes begin in utero and are coordinated with growth of the individual by a collaboration between the cells of cartilage and bone. The two principal cell types in bone are osteoblasts, which secrete bone matrix, and osteoclasts, which resorb bone. The coupled actions of both osteoblasts and osteoclasts are required for normal skeletal formation and maintenance, and for mineral homeostasis (Broadus, 1996, in Primer on the Metabolic Bone Diseases and Disorders of Mineral Metabolism, Favus (ed.), Lippincott-Raven, Philadelphia, pp. 57-63).

TRANCE (tumor necrosis factor-related, activation induced cytokine) is a member of the tumor necrosis factor family. It was initially discovered as a T-cell produced factor supporting the survival and activity of antigen presenting dendritic cells of the immune system (Anderson et al., 1997, Nature 390:175-179; Wong et al., 1997, J. Biol. Chem. 272:25190-25194; Wong et al., 1999, Mol. Cell 4:1041-1049). TRANCE has also been known as RANKL (receptor activator of NF-KB ligand). It was soon recognized that TRANCE was what had been known as osteoclast differentiation factor (ODF). ODF is produced by osteoblasts and plays an obligatory role in the formation and activation of osteoclasts, which are multinucleated, monocyte-macrophage lineage derived cells that carry out bone resorption (Yasuda et al., 1998, Proc. Natl. Acad. Sci. USA 95:3597-3602). The roles of TRANCE in the immune system and skeletal system were apparent in the phenotype of TRANCE-null (TRANCE knockout) mice. The knockout mice had severe osteopetrosis, lacked osteoclasts, failed to develop lymph nodes, and had defective T-cell and B-cell differentiation (Kong et al., 1999, Nature 397:315-323). Kong et al. referred to TRANCE as OPGL (steoprotegerin ligand).

TRANCE binding to its receptor (TRANCE-R, or RANK (receptor activator of NF-KB)) (a member of the tumor necrosis factor receptor family) activates the same intracellular signaling cascade in primary cultures of both dendritic cells and osteoclasts (Wong et al., 1999, Mol. Cell 4:1041-1049). The cytoplasmic domain of TRANCE-R is associated with TNF-receptor-associated factor 6 (TRAF6), which upon binding of TRANCE, enhances the kinase activity of c-src to activate the antiapoptotic serine/threonine kinase Akt/PKB, presumably thereby permitting the survival and activation of osteoclasts to resorb bone.

The invention is based on the discovery that TRANCE acts directly on cartilage-producing cells (chondrocytes). More particularly, TRANCE directly regulates the maturation of growth plate chondrocytes during endochondral ossification. In addition, it has been discovered that RANK and TRAF6 are expressed in precisely the regions of growing cartilage where many critical events occur in the transition from bone to cartilage, i.e., a zone of cartilage growth called the pre-hypertrophic zone.

Based on these discoveries, the invention features methods of treating a mammal, e.g., a human patient, having a disorder, e.g., a disease or condition, characterized by insufficient cartilage growth or insufficient skeletal growth, e.g., dwarfism, osteopetrosis, craniofacial-skeletal discrepancies, and bone or cartilage damage resulting from traumatic injury, surgery, osteoarthritis, or rheumatoid arthritis. The methods involve limiting or reducing TRANCE-dependent effects in chondrocytes. In one aspect, the invention provides a method of treating a disorder in a mammal having insufficient cartilage growth or insufficient skeletal growth by administering to the mammal an amount of a tumor necrosis factor-related activation induced cytokine (TRANCE)-inhibiting agent effective to increase cartilage growth or skeletal growth. In one embodiment, the method includes administering to the mammal an antisense molecule, e.g., an antisense nucleic acid, directed against a TRANCE RNA or a TRAF6 RNA. The antisense nucleic acid can be administered locally at a site of insufficient cartilage growth or insufficient skeletal growth. In another embodiment, the TRANCE-inhibiting agent is a TRANCE-binding molecule that sequesters TRANCE to form an inactive complex. The TRANCE-binding molecule can be, for example, an isolated RANK receptor, or a TRANCE-binding fragment thereof, or an anti-TRANCE antibody, or TRANCE-binding fragments thereof.

In another aspect, invention also features methods of treating a mammal having a disorder characterized by excessive cartilage growth or excessive skeletal growth, e.g., acromegaly, gigantism, exostosis cartilaginea, exostosis bursata, and multiple osteocartilaginous exostoses. Thus, the invention provides a method of treating a disorder in a mammal having excessive cartilage growth or excessive skeletal growth, comprising administering to the mammal an amount of a TRANCE-increasing agent effective to decrease cartilage growth or skeletal growth. In one embodiment, the TRANCE-increasing agent is a polypeptide comprising a tumor necrosis factor (TNF) domain of a TRANCE protein. The TRANCE protein can be from any mammal, e.g., a human or mouse TRANCE protein. In some embodiments, the polypeptide is locally administered to the mammal at a site of excessive cartilage growth or excessive skeletal growth. In other embodiments, the polypeptide is a polypeptide that includes amino acids 126-317 (SEQ ID NO:3), 137-317 (SEQ ID NO:4), 140-317 (SEQ ID NO:5), 145-317 (SEQ ID NO:6), 158-317 (SEQ ID NO:7) or 159-317 (SEQ ID NO:8) of human TRANCE (SEQ ID NO:2), but lacks the TRANCE cytoplasmic domain and transmembrane domain. In still other embodiments, the polypeptide consists of only amino acids 126-317, 137-317, 140-317, 145-317, 158-317 or 159-317 of human TRANCE.

In another embodiment, the TRANCE-increasing agent is a TRAF6 polypeptide. The TRAF6 polypeptide can be from any mammal, e.g., a human. The method can further include introducing a TRAF6 polypeptide into a chondrocyte at a site of excessive cartilage growth or excessive skeletal growth in the mammal. The TRAF6 polypeptide can be introduced indirectly by local administration of an expression vector including a TRAF6-encoding nucleotide sequence operably linked to one or more expression control sequences. Administration is such that the expression vector enters the chondrocyte and expresses the TRAF6 polypeptide in the chondrocyte. Alternatively, the TRAF6 polypeptide can be introduced directly. For example the TRAF6 polypeptide can be linked to a membrane translocation moiety to form a cell-permeating TRAF6, and administered at a site of insufficient cartilage growth or insufficient skeletal growth.

In another aspect, the invention features methods of promoting growth of cartilage in a mammal. In some embodiments, the method includes removing cartilage from a mammal; culturing the cartilage in vitro; contacting chondrocytes in the cartilage with a TRANCE inhibiting agent; and re-introducing the cartilage into the mammal. In specific embodiments, the TRANCE-inhibiting agent is, for example, a TRANCE antisense nucleic acid or a TRAF6 antisense nucleic acid. In yet another embodiment, the TRANCE-inhibiting agent is a TRANCE-binding molecule, e.g., an isolated RANK receptor, or a TRANCE-binding fragment thereof, or an anti-TRANCE antibody, or TRANCE-binding fragments thereof.

In another aspect, the invention features a method of diagnosing a cartilage disorder in a mammal. The method comprises obtaining a chondrocyte from the mammal; detecting a level of TRANCE, RANK, or TRAF6 in the chondrocyte, where a level of TRANCE, RANK, or TRAF6 that is elevated or reduced compared to a normal level indicates the presence of a cartilage disorder in the mammal.

In still another aspect, the invention features a method of identifying a candidate TRANCE-inhibiting compound. The method comprises obtaining a cultured, proliferating test chondrocyte; contacting the test chondrocyte with a test compound and a TRANCE polypeptide; and detecting proliferation in the test chondrocyte compared to a control chondrocyte contacted with a TRANCE polypeptide unaccompanied by the test compound, where an increase in proliferation indicates that the test compound is a candidate TRANCE-inhibiting compound. The chondrocyte can be any chondrocyte, e.g., a primary chondrocyte, a chondrocyte from a cultured chondrocyte cell line, a primary chondrocyte from a TRANCE null, transgenic non-human mammal, or a chondrocyte from a cultured chondrocyte cell line derived from a TRANCE null, transgenic non-human mammal.

In yet another aspect, the invention features a method of identifying a candidate TRANCE-increasing compound. The method comprises obtaining a cultured, proliferating test chondrocyte; contacting the test chondrocyte with a test compound and a TRANCE polypeptide; and detecting proliferation in the test chondrocyte compared to a control chondrocyte contacted with a TRANCE polypeptide unaccompanied by the test compound, where a decrease in proliferation indicates that the test compound is a candidate TRANCE-increasing compound. The chondrocyte can be any chondrocyte, e.g. a primary chondrocyte, a chondrocyte from a cultured chondrocyte cell line, a primary chondrocyte from a TRANCE null, transgenic non-human mammal, or a chondrocyte from a cultured chondrocyte cell line derived from a TRANCE null, transgenic non-human mammal.

Unless otherwise defined, all technical and scientific terms used herein have the same 10 meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a representation of the Human TRANCE splice variant 1 cDNA, GenBank accession # NM_{—}003701 (SEQ ID NO: 1). An example of a target region for antisense nucleic acids is shown italicized and underlined, with the translation start site in bold.

FIG. 2 is a representation of the Human TRANCE splice variant 1 amino acid sequence (SEQ ID NO:2).

FIG. 3 is a representation of the Human TRANCE splice variant 2 cDNA, GenBank Accession # NM_{—}033012 (SEQ ID NO:9). An example of a target region for antisense nucleic acids is shown italicized and underlined, with the translation start site in bold.

FIG. 4 is a representation of the Human TRANCE splice variant 2 amino acid sequence (SEQ ID NO:10).

FIG. 5 is a representation of the cDNA sequence of human TRANCE receptor (RANK), GenBank accession #NM_{—}003839 (SEQ ID NO:11).

FIG. 6 is a representation of the Human TRANCE receptor (RANK) amino acid sequence (SEQ ID NO:12).

FIG. 7 is a representation of the Human TRAF6 cDNA, GenBank Accession # XM_{—}006284 (SEQ ID NO: 13). An example of a target region for antisense nucleic acids is shown italicized and underlined, with the translation start site in bold.

FIG. 8 is a representation of the Human TRAF6 amino acid sequence (SEQ ID NO: 14).

FIGS. 9A-D is a representation of an alignment of TRANCE cDNA sequences from mouse (SEQ ID NO:15) and human splice variant 1 (SEQ ID NO:1). The symbol (*) indicates an identity, and the symbol (−) indicates a gap.

FIG. 10 is a representation of an alignment of TRANCE amino acid sequences from mouse (SEQ ID NO:16) and human splice variant 1 (SEQ ID NO:2). The symbol (*) indicates an identity; the symbol (−) indicates a gap; the symbol (.) indicates non-conservative substitution; and the symbol (:) indicates conservative substitution.

FIG. 11 is a representation of the TNF core domain of human TRANCE, beginning at amino acid # 159 and continuing to the carboxy terminus (SEQ ID NO:8). Below the sequence are some examples of conservative amino acid substitutions that should not substantially alter the biological activity of TRANCE.

### DETAILED DESCRIPTION

TRANCE directly regulates the maturation, i.e., differentiation, of growth plate chondrocytes during endochondral ossification. TRANCE acts in growth plate chondrocytes by operating directly through a RANK and TRAF6-mediated pathway present in the chondrocytes. Various defects and disorders in cartilage maturation and bone growth result from abnormal, i.e., excessive or insufficient, TRANCE activity mediated by RANK and TRAF6 in chondrocytes. Based on these discoveries, the invention includes various therapeutic, diagnostic, and screening methods.

Therapeutic Methods

The invention provides therapeutic methods that fall into two basic categories: inhibiting (e.g., limiting or reducing) TRANCE activity, and increasing, e.g., supplementing or providing TRANCE activity where there is insufficient or no TRANCE activity. Whether TRANCE activity is inhibited or increased depends on the disease, disorder, or condition being treated.

Inhibiting TRANCE Activity

In some embodiments, the invention provides for treating a disorder characterized by insufficient cartilage growth or insufficient skeletal growth, e.g., dwarfism, osteopetrosis, craniofacial-skeletal discrepancies, and bone or cartilage damage resulting from traumatic injury, surgery, osteoarthritis, or rheumatoid arthritis. In general, to treat such a disorder or condition characterized by insufficient cartilage growth, cellular TRANCE activity is limited or reduced by the administration of a TRANCE-inhibiting agent. One such TRANCE-inhibiting agent is an antisense oligonucleotide directed against TRANCE mRNA. The antisense oligonucleotide interferes with translation of TRANCE mRNA, thereby inhibiting expression of TRANCE polypeptides in cells.

An antisense nucleic acid effective to inhibit expression of an endogenous TRANCE gene can include a nucleotide sequence complementary to an entire TRANCE RNA or only a portion of the RNA. On one hand, the antisense nucleic acid needs to be long enough to hybridize effectively with the TRANCE RNA. Therefore, the minimum length is approximately 10, 11, 12, 13, 14, or 15 nucleotides. On the other hand, as length increases beyond about 150 nucleotides, effectiveness at inhibiting translation increases only marginally, while difficulty in introducing the antisense nucleic acid into target cells may increase significantly. In view of these considerations, a preferred length for the antisense nucleic acid is from about 15 to about 150 nucleotides, e.g., 20, 25, 30, 35, 40, 45, 50, 60, 70, or 80 nucleotides. The antisense nucleic acid can be complementary to a coding region of TRANCE mRNA or a 5′ or 3′ non-coding region of a TRANCE mRNA (or both). One effective approach is to design the antisense nucleic acid to be complementary to a region on both sides of the translation start site of the TRANCE mRNA, e.g., complementary to a region including about nucleotide −10 to +10 of the TRANCE mRNA.

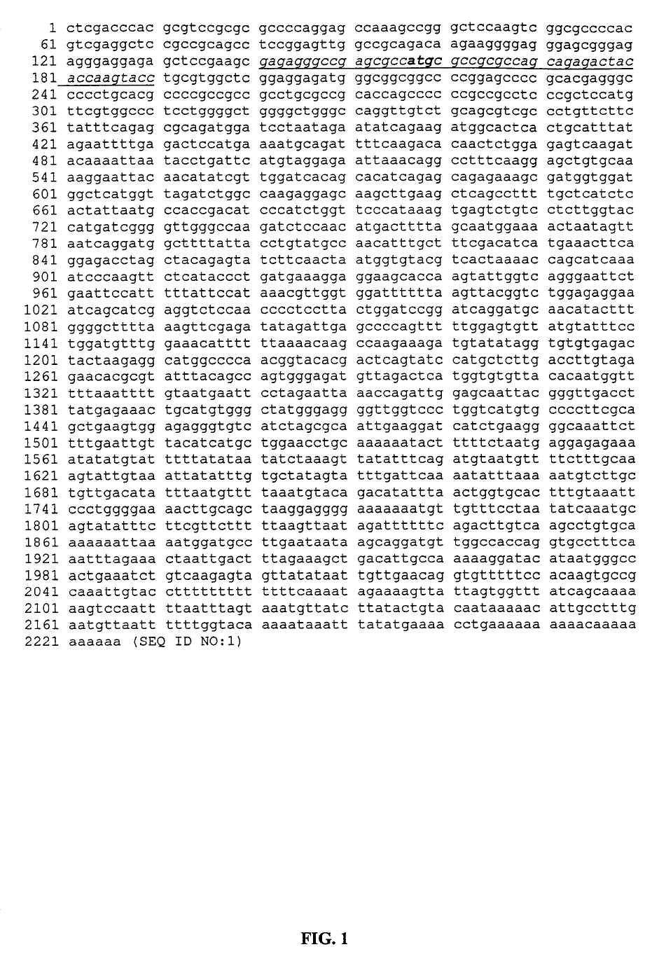

For example, human TRANCE antisense oligonucleotides that consist of 24 to 50 nucleotides encompassing the translation initiation codon can be used to inhibit TRANCE activity. In Homo sapiens tumor necrosis factor (ligand) superfamily, member 11 (TNFSF11), mRNA splice variant 1 (GenBank Accession # NM_{—}003701.2), this would include the region from nucleotides 141 to 190 (SEQ ID NO:17), including the start codon at nucleotide number 157 (see FIG. 1). In mRNA splice variant 2 (GenBank Accession # NM_{—}033012.1), this would include nucleotides 81 to 131 (SEQ ID NO:18), including the start codon at nucleotide number 95 (see FIG. 3).

TRANCE-dependent effects in chondrocytes also can be effectively limited or reduced by administering an antisense molecule directed against TRAF6 mRNA. By inhibiting cellular expression of TRAF6 polypeptides, which mediate TRANCE activity in at least some TRANCE-dependent effects in chondrocytes, TRANCE activity is inhibited indirectly. Thus, antisense oligonucleotides strand directed against TRAF6 mRNA is also a TRANCE-inhibiting agent.

For example, human TRAF6 antisense oligonucleotides that consist of 24 to 50 nucleotides encompassing the translation initiation codon can be used to indirectly inhibit TRANCE activity. In Homo sapiens TNF receptor-associated factor 6 (TRAF6), mRNA (GenBank Accession # XM_{—}006284.3), this would include the region from nucleotides 208 to 258 (SEQ ID NO:19), including the start codon at nucleotide 222.

The antisense nucleic acid can be chemically synthesized, e.g., using a commercial nucleic acid synthesizer according to the vendor's instructions. Alternatively, the antisense nucleic acids can be produced using recombinant DNA techniques. An antisense nucleic acid can incorporate only naturally occurring nucleotides. Alternatively, it can incorporate variously modified nucleotides or nucleotide analogs to increase its in vivo half-life or to increase the stability of the duplex formed between the antisense molecule and its target RNA. Examples of nucleotide analogs include phosphorothioate derivatives and acridine-substituted nucleotides. Given the description of the targets and sequences, the design and production of suitable antisense molecules is within ordinary skill in the art. For guidance concerning antisense nucleic acids, see, e.g., Goodchild, “Inhibition of Gene Expression by Oligonucleotides,” in Topics in Molecular and Structural Biology, Vol. 12: Oligodeoxynucleotides (Cohen, ed.), MacMillan Press, London, pp. 53-77.

Delivery of antisense oligonucleotides can be accomplished by any method known to those of skill in the art. For example, delivery of antisense oligonucleotides for cell culture and/or ex vivo work can be performed by standard methods such as the liposome method or simply by addition of membrane-permeable oligonucleotides. To resist nuclease degradation, chemical modifications such as phosphorothionate backbones can be incorporated into the molecule.

Delivery of antisense oligonucleotides for in vivo applications can be accomplished, for example, via local injection of the antisense oligonucleotides at an affected site. This method has previously been demonstrated for psoriasis growth inhibition and for cytomegalovirus inhibition. See, for example, Wraight et al., (2001). Pharmacol Ther. Apr; 90(1):89-104.; Anderson, et al., (1996) Antimicrob Agents Chemother 40: 2004-2011; and Crooke et al., J Pharmacol Exp Ther 277: 923-937.

As another example, the antisense oligonucleotide or a plasmid encoding it can be mixed with collagen or other biocompatible polymer and implanted to achieve sustained release into the cartilage matrix. See, for example, Kyriakides et al., (2001) Mol. Ther. 3(6):842-9.

TRANCE-dependent effects in chondrocytes also can be effectively limited or reduced by locally administering a soluble TRANCE-binding molecule or agent, e.g., a molecule or agent which can sequester TRANCE in an inactive complex, into an extracellular matrix surrounding the chondrocytes. The soluble TRANCE-binding molecule competes with RANK, i.e., the physiological TRANCE receptor, anchored on the surfaces of the chondrocytes, thereby sequestering TRANCE. An example of a soluble TRANCE-binding molecule is osteoprotegerin (OPG; a soluble form of RANK), which is secreted from some types of mammalian cells. See, e.g., Simonet et al., 1997, Cell 89:309-319. Thus, soluble TRANCE-binding molecules are TRANCE inhibiting agents.

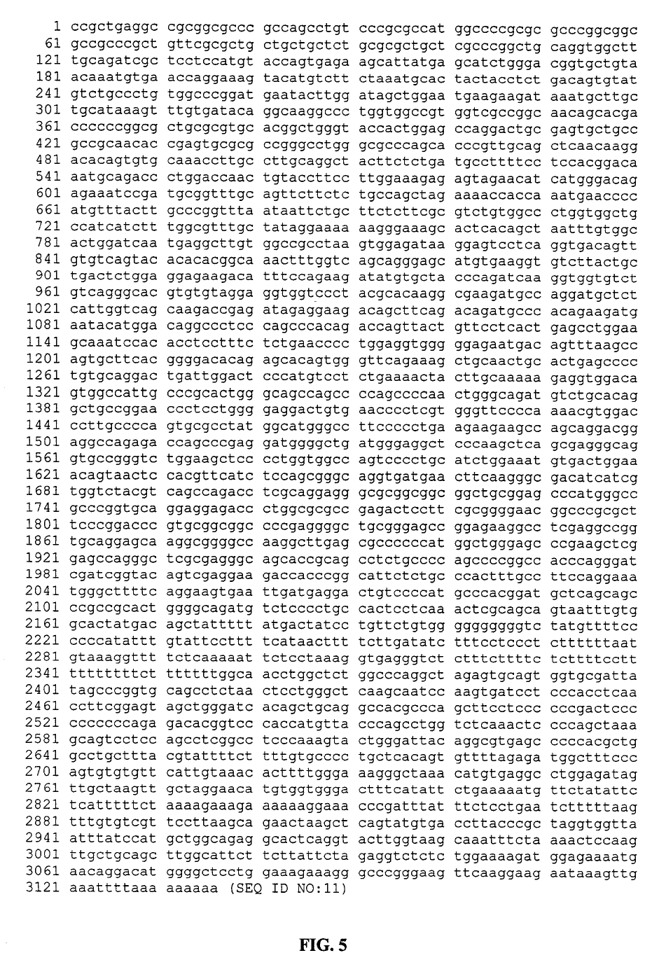

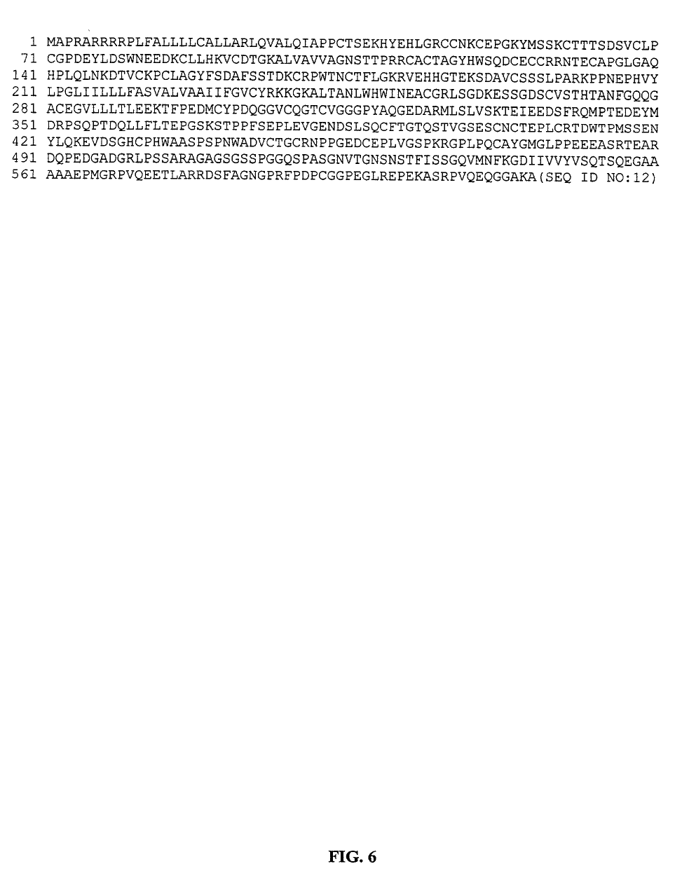

As another example, the soluble TRANCE-binding molecule can be an isolated RANK receptor. FIG. 5 depicts the cDNA sequence of human TRANCE receptor (RANK), GenBank accession #NM_{—}003839 (SEQ ID NO:11), and FIG. 6 depicts the RANK amino acid sequence (SEQ ID NO:12). The soluble TRANCE-binding molecule can also be a TRANCE-binding fragment of a RANK receptor, e.g. a fragment of a RANK receptor, which retains the ability to interact with a TRANCE molecule. The RANK receptor can be a natural RANK receptor, e.g., RANK receptors harvested directly from chondrocytes. The skilled artisan will appreciate that TRANCE-binding fragments of the natural RANK receptor can be created, for example, by digestion with appropriate enzymes. Similarly, the RANK receptor can be a recombinant or synthetic RANK receptor. It is contemplated by the present invention that nucleic acid sequences encoding a RANK receptor (e.g., Accession No: AF018253), or TRANCE-binding fragments thereof, can be linked to an appropriate promoter for production of RANK receptors or TRANCE-binding fragments in any given expression system. Alternatively, whole RANK receptors, e.g. whole recombinant RANK receptors, can be produced in an expression system, and then subjected to an appropriate process, e.g. enzymatic digestion, to create TRANCE-binding fragments of RANK receptors.

As yet another example, the TRANCE-binding molecule (TRANCE-inhibiting agent) can be an antibody, e.g., an anti-TRANCE antibody, or a TRANCE-binding fragment thereof. The anti-TRANCE antibody can be a polyclonal or a monoclonal antibody. Alternatively, the antibody can be recombinantly produced, e.g., produced by phage display or by combinatorial methods as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982.

As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917). Each VH and VL is composed of three CDR's and four FRs, arranged from amino-terminus to carboxy-terminus in the following order: FR1, CDR1, FR2, CDR2, FR3, CDR3, FR4.

An anti-TRANCE or anti-RANK antibody can further include a heavy and light chain constant region, to thereby form a heavy and light immunoglobulin chain, respectively. The antibody can be a tetramer of two heavy imunoglobulin chains and two light immunoglobulin chains, wherein the heavy and light immunoglobulin chains are inter-connected by, e.g., disulfide bonds. The heavy chain constant region is comprised of three domains, CH1, CH2, and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

A “TRANCE-binding fragment,” or a “RANK-binding fragment” of an antibody refers to one or more fragments of a full-length antibody that retain the ability to specifically bind to TRANCE or a portion thereof or RANK or a portion thereof. “Specifically binds” means that an antibody or ligand binds to a particular target to the substantial exclusion of other substances. Examples of TRANCE- or RANK-binding fragments of an anti-TRANCE or anti-RANK antibody include, but are not limited to: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are encoded by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “TRANCE-binding fragment” or “RANK-binding fragment” of an antibody. These antibody fragments can be obtained using conventional techniques known to those with skill in the art.

The anti-TRANCE or anti-RANK antibody can be a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel, donkey, porcine, or fowl antibody.

An anti-TRANCE or anti-RANK antibody can be one in which the variable region, or a portion thereof, e.g., the CDRs, are generated in a non-human organism, e.g., a rat or mouse. The anti-TRANCE or anti-RANK antibody can also be, for example, chimeric, CDR-grafted, or humanized antibodies. The anti-TRANCE or anti-RANK antibodies can also be generated in a non-human organism, e.g., a rat or mouse, and then modified, e.g., in the variable framework or constant region, to decrease antigenicity in a human.

Another approach to inhibiting TRANCE-dependent effects in chondrocytes is the administration of a TRANCE antagonist that binds to (blocks) RANK on chondrocyte surfaces, without triggering signal transduction. The soluble RANK-binding molecule competes with TRANCE thereby sequestering RANK. Such RANK blockers can be identified using a screening method described below. Alternatively, the TRANCE antagonist can be an anti-RANK antibody, or fragment thereof, as described above.

Agents, e.g., antibodies and/or compounds, that are used to inhibit the interaction of TRANCE with its receptor, can be specifically targeted to the second cysteine-rich domain (CRD2) domain of the receptor (RANK), where TRANCE and RANK have direct contact. Alternatively, TRANCE binding to its receptor can be blocked by agents that bind to the trimer-forming interface on the TRANCE monomer, or that mimic the receptor contact sites along CRD2 in the receptor. See, e.g., Locksley et al., Cell (2001) 104(4):487-501.

Increasing TRANCE Activity

In some embodiments, the invention is used to treat a disease, disorder, or condition characterized by excessive cartilage growth or excessive skeletal growth. Examples of such diseases, disorders, or conditions include acromegaly, gigantism, exostosis cartilaginea, exostosis bursata, and multiple osteocartilaginous exostoses. In some situations, excessive cartilage growth is reflected in excessive bone growth, e.g., gigantism. In general, to treat a disease, disorder, or condition characterized by excessive cartilage growth or excessive skeletal growth, TRANCE activity and/or TRAF6 activity is therapeutically increased in chondrocytes in a location where cartilage growth or skeletal growth is insufficient by administering a TRANCE-increasing agent. Increased TRANCE activity or TRAF6 activity (or both) can be accomplished in various ways, as described herein.

For example, new or supplemental TRANCE activity can be provided in vivo by local administration of a recombinant, soluble TRANCE polypeptide (a type of TRANCE-increasing agent) (SEQ ID NO:2) at a site of excessive cartilage growth or excessive skeletal growth. For example, a therapeutically effective amount of a human TRANCE polypeptide dissolved in a pharmaceutically acceptable medium can be injected into a joint, e.g., a knee, of a human patient.

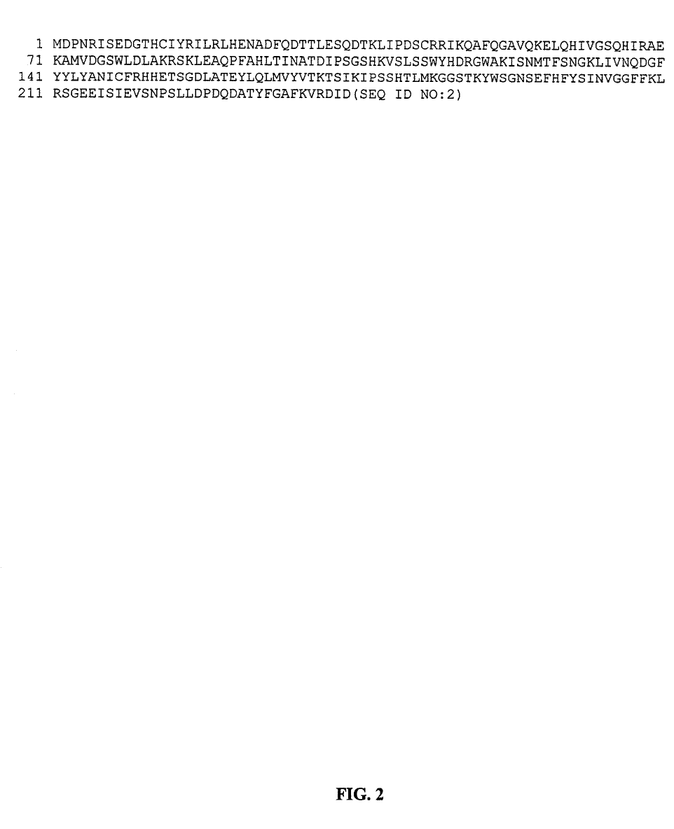

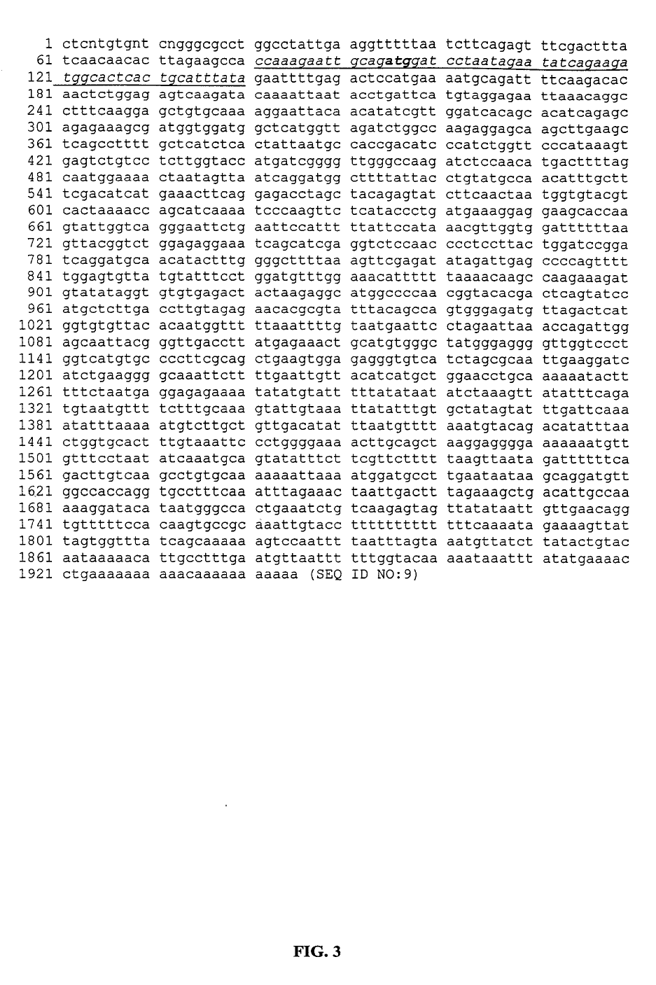

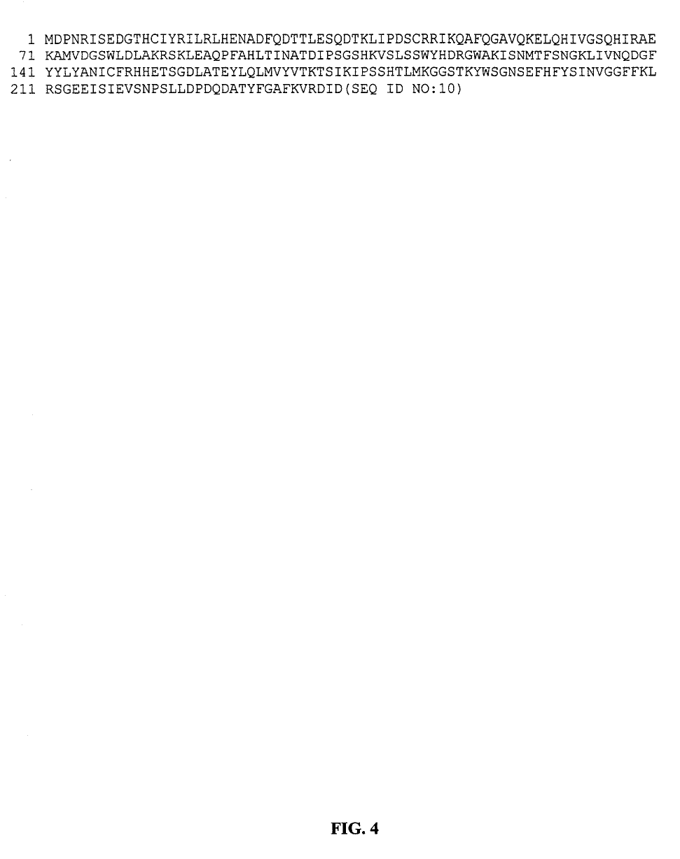

FIG. 2 depicts the Human TRANCE splice variant 1 amino acid sequence (SEQ ID NO:2), and FIG. 4 depicts the Human TRANCE splice variant 2 amino acid sequence (SEQ ID NO:10). Further examples of suitable soluble TRANCE polypeptides include the following: a polypeptide that includes amino acids 126-317 (SEQ ID NO:3), 137-317 (SEQ ID NO:4), 140-317 (SEQ ID NO:5), 145-317 (SEQ ID NO:6), 158-317 (SEQ ID NO:7), or 159-317 (SEQ ID NO:8) of human TRANCE (SEQ ID NO:2), but lacks the TRANCE cytoplasmic domain and transmembrane domain (see, e.g., Lacey et al., 1998, Cell 93:165-176). Such polypeptides can be used “as is” or modified. Examples of modifications include derivation of amino acid side chains, glycosylation, conservative amino acid substitutions, and chemical conjugation or fusion to non-TRANCE polypeptide moieties.

One example of a useful polypeptide is a soluble, C-terminal fragment (approximately 31 kD), which contains the TNF domain of the TRANCE polypeptide. This polypeptide can be produced by cleavage of full-length TRANCE, e.g., from mammalian cells expressing TRANCE. This soluble, TNF domain-containing TRANCE polypeptide fragment exerts a therapeutic effect by binding to a cell surface receptor on chondrocytes, e.g., RANK. Therefore, when administered as described herein, entry of the soluble, TNF domain-containing TRANCE polypeptide fragment into chondrocytes or other cells, i.e., crossing of an intact cytoplasmic membrane, is not necessary to achieve the desired therapeutic effect.

As another example, TRANCE activity can be increased by supplementing the supply of TRAF6, a TRANCE-triggered downstream signaling factor, in chondrocytes. Thus, TRAF6 polypeptides can serve as TRANCE-increasing agents. A therapeutically effective amount of TRAF6 can be introduced into chondrocytes directly or indirectly. Direct introduction of TRAF6 into chondrocytes involves administration of TRAF6 polypeptide. A directly administered TRAF6 polypeptide can be covalently linked to a membrane translocation moiety using known techniques, which facilitates entry of the TRAF6 polypeptide into the chondrocyte from the extracellular matrix. Examples of useful membrane translocation moieties include the internalization peptide sequence derived from Antennapedia (Bonfanti et al., Cancer Res. 57:1442-1446) or from an HIV tat peptide (U.S. Pat. No. 5,652,122), either of which can be fused to the N-terminus of the TRAF6 polypeptide. Alternatively, the TRAF6 polypeptide can be expressed in the chondrocyte following introduction of a TRAF6 polypeptide-encoding DNA, e.g., in a conventional expression vector.

A TRANCE polypeptide or TRAF6 polypeptide can be introduced indirectly into a mammal, e.g., a mouse, rat, rabbit, or human patient, by expressing within chondrocytes of the mammal a nucleic acid construct containing a nucleotide sequence encoding a TRANCE polypeptide or TRAF6 polypeptide. Any expression vector suitable for transfecting a mammalian cell can be used in the invention. The nucleic acid construct can be derived from a non-replicating linear or circular DNA or RNA vector, or from an autonomously replicating plasmid or viral vector. Methods for constructing suitable expression vectors are known in the art, and useful materials are commercially available. A number of vectors suitable for stable transfection of mammalian cells are available to the public, see, e.g., Pouwels et al. (supra); methods for constructing such cell lines are also publicly known, e.g., in Ausubel et al. (supra). In one example, DNA encoding the protein is cloned into an expression vector that includes the dihydrofolate reductase (DHFR) gene. Integration of the plasmid and the TRANCE or TRAF6 polypeptide-encoding gene into the host cell chromosome is selected for by including 0.01-300 μM methotrexate in the cell culture medium (as described in Ausubel et al., supra). This dominant selection can be accomplished in most cell types.

Regardless of TRANCE polypeptide or TRAF6 polypeptide delivery methods used, response to therapeutic treatment according to the invention can be measured by various suitable methods known in the art. For example, conventional radiography can be used to assess skeletal growth or repair following treatment. Other methods of assessing response to therapeutic treatment include nuclear magnetic resonance (NMR) imaging, tomography, and measurements of bone mineral density.

In Vitro and Ex Vivo Therapeutic Methods

The invention includes methods of inhibiting chondrocyte differentiation and/or promoting growth of cartilage in vitro. In such methods, an effective amount of a TRANCE antisense nucleic acid, a TRAF6 antisense nucleic acid, or a TRANCE-binding agent, is added to the growth medium of cultured chondrocytes or cultured cartilage-containing chondrocytes.

In some embodiments of the invention, chondrocytes or cartilage are removed from a mammal, e.g., a human patient, treated with a TRANCE antisense nucleic acid, a TRAF6 antisense nucleic acid, or a TRANCE-binding agent in vitro to inhibit chondrocyte differentiation and/or to promote cartilage growth, and then re-introduced (re-implanted) at a location in the body of the mammal where cartilage growth or proliferating chondrocytes are needed. In vitro culture of chondrocytes and surgical implantation of chondrocytes and/or cartilage is known in the art. See, e.g., Kuettner, U.S. Pat. No. 4,356,261; Tubo et al., U.S. Pat. No. 5,786,217; and Tubo et al., U.S. Pat. No. 5,723,331.

Diagnostic Methods

Various diseases, disorders, or conditions involving excessive cartilage growth or insufficient cartilage growth can be diagnosed, assessed or studied in a mammal, e.g., a human patient, according to the invention. This is accomplished by detecting an elevated level or a reduced level of TRANCE, RANK, or TRAF6 in a sample of chondrocytes from the mammal. In general, overexpression of TRANCE or TRAF6 is associated with insufficient cartilage growth, and an abnormally low amount of functional (biologically active) TRANCE, RANK, or TRAF6 polypeptides in an affected tissue is associated with excessive cartilage growth. An abnormally low level of active TRANCE, RANK, or TRAF6 polypeptides can result from any of various factors, including defects in the respective genomic coding sequences, defects in the respective expression control elements, altered cellular localization of the RNAs or polypeptides, deficient processing or translation of the RNA, and increased degradation of the polypeptides. Therefore, diagnostic methods can include one or more of the following: examination of genomic TRANCE, RANK, or TRAF6 coding sequences in chondrocytes; examination of TRANCE, RANK, or TRAF6 expression control sequences in chondrocytes; measurement of TRANCE, RANK, or TRAF6 mRNA levels in chondrocytes, e.g., by Northern blot or dot blot; detection of TRANCE, RANK, or TRAF6 RNA and/or polypeptide localization in chondrocytes, e.g., by in situ hybridization; and measurement of TRANCE, RANK, or TRAF6 polypeptide levels in chondrocytes or cartilage, e.g., by ELISA or Western blot.

For example, isolated TRANCE, RANK, or TRAF6 mRNA can be used in hybridization or amplification assays that include, but are not limited to, Southern or Northern analyses, polymerase chain reaction analyses and probe arrays. One diagnostic method for the detection of TRANCE, RANK, or TRAF6 mRNA levels involves contacting isolated mRNA with a nucleic acid molecule (probe) that can hybridize to TRANCE, RANK, or TRAF6 mRNA encoded by the gene being detected. The nucleic acid probe can be, for example, a full-length TRANCE, RANK, or TRAF6 nucleic acid, or a portion thereof, which specifically hybridizes under stringent conditions to TRANCE, RANK, or TRAF6 mRNA or genomic DNA. In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the isolated mRNA (or cDNA) is contacted with the probes, for example, in a two-dimensional gene chip array. A skilled artisan can adapt known mRNA detection methods for use in detecting the level of mRNA encoded by the TRANCE, RANK, or TRAF6 genes.

The level of mRNA in a sample that is encoded by TRANCE, RANK, or TRAF6 genes can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al., (1989), Proc. Natl. Acad. Sci. USA 86:1173-1177), Q-Beta Replicase (Lizardi et al., (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes TRANCE, RANK, or TRAF6.

A variety of methods can be used to determine the level of TRANCE, RANK, or TRAF6 polypeptides in a sample. Such methods include contacting the sample with an agent that specifically binds to the protein, e.g., an antibody, to evaluate the level of protein in the sample. The antibody can bear a detectable marker, e.g., radioactive or fluorescence markers. Antibodies can be polyclonal, or more preferably, monoclonal. An intact antibody, or a fragment thereof (e.g., Fab or F(ab′)₂) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Techniques for detection of TRANCE, RANK, or TRAF6 polypeptides can include enzyme linked immunosorbent assays (ELISAs), immunoprecipitations, immunofluorescence, enzyme immunoassay (EIA), radioimmunoassay (RIA), and Western blot analysis.

A suitable sample to be assayed for TRANCE, RANK, or TRAF6 RNA can be obtained by any technique known to those skilled in the art. The term “sample” can include tissues, cells, and biological fluids isolated from a subject, as well as tissues, cells and fluids present within a subject. For example, a sample of chondrocytes and/or cartilage can be obtained by conventional surgical biopsy techniques. Suitable assay techniques, e.g., PCR, nucleotide sequence analysis, Southern and Northern blot analysis, dot blot analysis, in situ hybridization, ELISA, and Western blot analysis are known in the art and can be carried out without undue experimentation.

The skilled artisan will appreciate that “normal” or control levels of TRANCE, RANK, or TRAF6 RNA, DNA or protein, e.g., in a normal chondrocyte, can be determined by any method known in the art. For example, it is common practice to determine a normal level of RNA, DNA, or protein by testing a dilution series, and comparing the data to experimental results. Experimental results can be expressed in any art-accepted form, e.g., as a percentage of the normal or control amount.

Screening Methods

The invention also provides in vitro screening methods for identifying compounds, e.g., small organic or inorganic molecules (M.W. less than 1,000 Da), oligopeptides, oligonucleotides, or carbohydrates that modulate TRANCE-mediated effects on chondrocytes. In some embodiments, cultured chondrocytes employed in the in vitro screening assay are derived from an experimental non-human mammal, e.g., a mouse, rat, or rabbit. In other embodiments, the cultured chondrocytes are derived from human tissue.

One convenient indicator of TRANCE action on chondrocytes is rate of proliferation of the chondrocytes. A test compound that enhances TRANCE action on chondrocytes, when contacted with proliferating chondrocytes in combination with TRANCE, decreases chondrocyte proliferation, relative to controls treated with TRANCE unaccompanied by the candidate compound. A test compound with such activity is thus a candidate TRANCE increasing compound. Conversely, a test compound that inhibits TRANCE action on chondrocytes, when contacted with the chondrocytes in combination with TRANCE, will increase chondrocyte proliferation (relative to TRANCE-only controls). Such a test compound is thus a candidate TRANCE inhibiting compound.

A “test compound” can be any chemical compound, for example, a macromolecule (e.g., a polypeptide, a protein complex, or a nucleic acid) or a small molecule (e.g., an amino acid, a nucleotide, an organic or inorganic compound). The test compound can have a formula weight of less than about 10,000 grams per mole, less than 5,000 grams per mole, less than 1,000 grams per mole, or less than about 500 grams per mole. The test compound can be naturally occurring (e.g., an herb or a natural product), synthetic, or can include both natural and synthetic components. Examples of small molecules include peptides, peptidomimetics (e.g., peptoids), amino acids, amino acid analogs, polynucleotides, polynucleotide analogs, nucleotides, nucleotide analogs, and organic or inorganic compounds, e.g., heteroorganic or organometallic compounds.

Changes in chondrocyte proliferation in vitro can be detected readily using known techniques. One approach is visual counting of cells. For example, rate of cell division can be measured by counting the difference in cell number at two time points, taking the log₂ of the difference, and dividing that value by the time elapsed between the two time points. Alternatively, a chondrocyte's rate of DNA synthesis can be measured, for example, by growing the cell in a medium containing ³H-thymidine and measuring incorporation of the ³H-thymidine into chondrocyte DNA at two time points. The rate of ³H-thymidine incorporation into the cell's DNA is directly proportional to the rate of DNA synthesis, which indicates rate of cell proliferation. Alternatively, incorporation of bromodeoxyuridine into chondrocyte DNA can measured.

Another convenient indicator of TRANCE action on chondrocytes is differentiation (maturation) of chondrocytes. One change that occurs in chondrocyte maturation is a switch from production of type II collagen to type X collagen. Therefore, detection of type X collagen production (or an increase in type X collagen production) in response to a test compound plus a TRANCE polypeptide, relative to TRANCE unaccompanied by the test compound, indicates that the compound increases TRANCE activity in the chondrocytes. Conversely, a decrease in type X collagen production in response to a test compound plus a TRANCE polypeptide, relative to TRANCE unaccompanied by the test compound, indicates that the compound inhibits TRANCE activity in the chondrocytes.

Test compounds can be screened individually or in parallel. An example of parallel screening is a high throughput drug screen of large libraries of chemicals. Such libraries of test compounds can be generated or purchased, e.g., from Chembridge Corp., San Diego, Calif. Libraries can be designed to cover a diverse range of compounds. For example, a library can include 500, 1000, 10,000, 50,000, or 100,000 or more unique compounds. Alternatively, prior experimentation and anecdotal evidence can suggest a class or category of compounds of enhanced potential. A library can be designed and synthesized to cover such a class of chemicals.

Examples of methods for the synthesis of molecular libraries can be found in the literature, for example in: DeWitt et al (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al (1994) Angew. Chem. Int. Ed. Engl. 33:2061; and Gallop et al (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992)Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

A cultured chondrocyte can be derived from a cell line, such as cell line ATDC5 (RIKEN cell bank, Japan). In some embodiments, detecting increased differentiation includes detecting expression of type X collagen. Detecting expression of type X collagen can include, e.g., reverse transcription PCR using collagen type-specific primers, or immunodetection using an antibody specific for collagen type X.

Primary cultures of mammalian chondrocytes useful for TRANCE screening assays can be performed using costochondral-derived cells according to the method of Boyan, et al. (1988, Bone 9:185-194). Resting and growth zones are dissected from rib cages of rats (100-150 g) and incubated overnight in DMEM. They are washed in HBSS and then sequentially digested at 37° with 1% trypsin (Sigma) for 1 hour and collagenase (Worthington, Type II) at 0.02% for 3 hours. Cells are then separated from debris by filtration through 40-mesh nylon, pelleted for 5 minutes at 500 rcf, resuspended in DMEM. Cell viability is assessed by dye exclusion, and the cells are plated in DMEM supplemented with 10% fetal bovine serum, 50 mg/ml ascorbate, and antibiotics. Cells are plated at 10,000 to 25,000 per cm² in multi-well culture dishes and incubated in a humidified, 5% CO₂ atmosphere at 37°. Alternatively, xyphoid cartilage from adult rats may be a suitable source tissue for primary cultures. See Rani, 1999, Dev. Dyn. 214:26-33. Cultures are maintained for a maximum of 3 passages. Although the foregoing is a description of primary chondrocyte cultures from rat source tissues, these techniques can be adapted readily for primary culture of chondrocytes from suitable tissue of other mammals, e.g., mice, rabbits and humans.

The presence of col II and col X mRNA in Northern blots or RT-PCR of RNA extracts (Trizol, Life Technologies) can be used to monitor chondrocyte phenotype. Total protein extracts can be made from some cultures by homogenizing in 8M urea containing 2% b-mercaptoethanol and 2% Non-Idet P40, as described in Odgren, 1996, J. Cell Sci. 109:2253-2264. These can be tested by Western blot for the presence and levels of TRANCE signaling molecules RANK and TRAF-6.

Compounds that alter chondrocyte proliferation or differentiation in an initial screen can be considered candidate compounds, e.g., candidate TRANCE-inhibiting or TRANCE-increasing compounds. Candidate compounds can be retested, e.g. on chondrocytes, e.g., in vitro, or tested on animals, e.g., animals that are models for abnormal (excessive or insufficient) cartilage or skeletal growth. Candidate compounds that are positive in a retest can be considered “lead” compounds to be further optimized and derivatized, or may be useful therapeutic or diagnostic compounds themselves.

Once a lead compound has been identified, standard principles of medicinal chemistry can be used to produce derivatives of the compound. Derivatives can be screened for improved pharmacological properties, for example, efficacy, pharmaco-kinetics, stability, solubility, and clearance. The moieties responsible for a compound's activity in the assays described above can be delineated by examination of structure-activity relationships (SAR) as is commonly practiced in the art. A person of ordinary skill in pharmaceutical chemistry could modify moieties on a lead compound and measure the effects of the modification on the efficacy of the compound to thereby produce derivatives with increased potency. For an example, see Nagarajan et al. (1988) J. Antibiot. 41: 1430-8. Furthermore, if the biochemical target of the lead compound is known or determined, the structure of the target and the lead compound can inform the design and optimization of derivatives. Molecular modeling software is commercially available (e.g., Molecular Simulations, Inc.) for this purpose.

TRANCE Polypeptides

As used herein, “the human TRANCE amino acid sequence” means the full-length human TRANCE amino acid sequence found in Lacey et al., 1998, Cell 93:165-176. A “TNF domain-containing TRANCE polypeptide” is a polypeptide that contains a TNF domain of a TRANCE protein. A “TNF domain” of TRANCE includes the TNF core domain of a TRANCE protein, e.g. the TNF core domain of human TRANCE that begins at about amino acid 159 and extends to about amino acid 317 of the human TRANCE protein.

Soluble, TNF domain-containing TRANCE polypeptides, e.g., polypeptides including amino acids 126-317 (SEQ ID NO:3), 137-317 (SEQ ID NO:4), 140-317 (SEQ ID NO:5), 145-317 (SEQ ID NO:6), 158-317 (SEQ ID NO:7), or 159-317 (SEQ ID NO:8) of the human TRANCE amino acid sequence (SEQ ID NO:2), are useful in methods of the invention. In addition, nucleic acids encoding full-length TRANCE polypeptides or fragments thereof are useful in the methods of the invention. TRANCE polypeptides and nucleic acids encoding them are readily obtained by one of ordinary skill in the art without undue experimentation. For example, the complete amino acid sequences of a mouse TRANCE polypeptide and a human TRANCE polypeptide, and methods of making soluble, TNF domain-containing TRANCE polypeptides (fragments) are found in Lacey et al. (supra). The complete amino acid sequence of the human TRANCE polypeptide is found in Anderson et al., 1997, Nature 390:175-179. The complete nucleotide sequence of a full-length human TRANCE cDNA is available from GenBank (Accession No. AF019047). A nucleic acid encoding a mammalian, e.g., human, TRANCE amino acid sequence can be amplified from human cDNA by conventional PCR techniques, using primers upstream and downstream of the coding sequence.

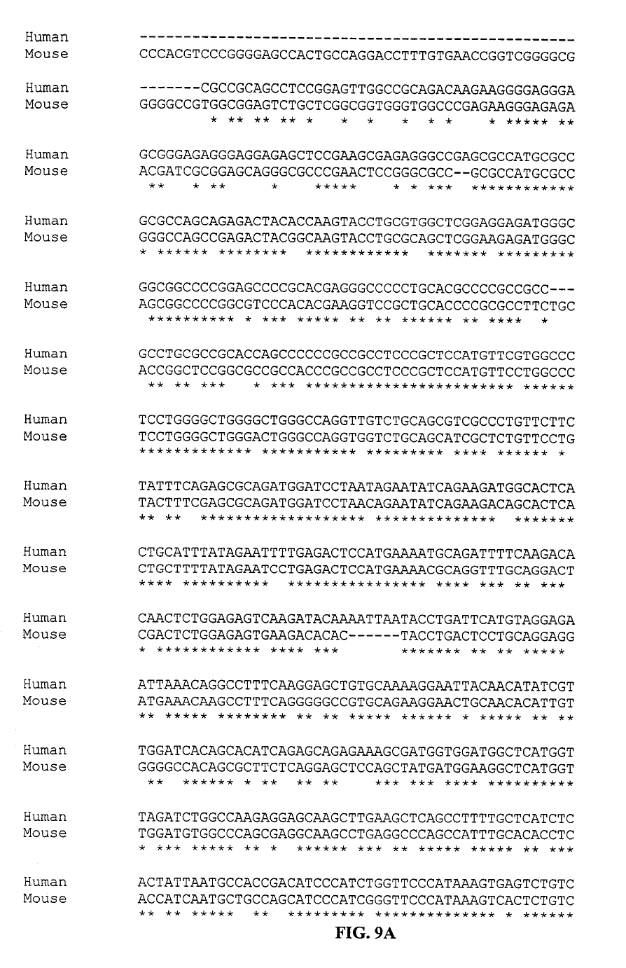

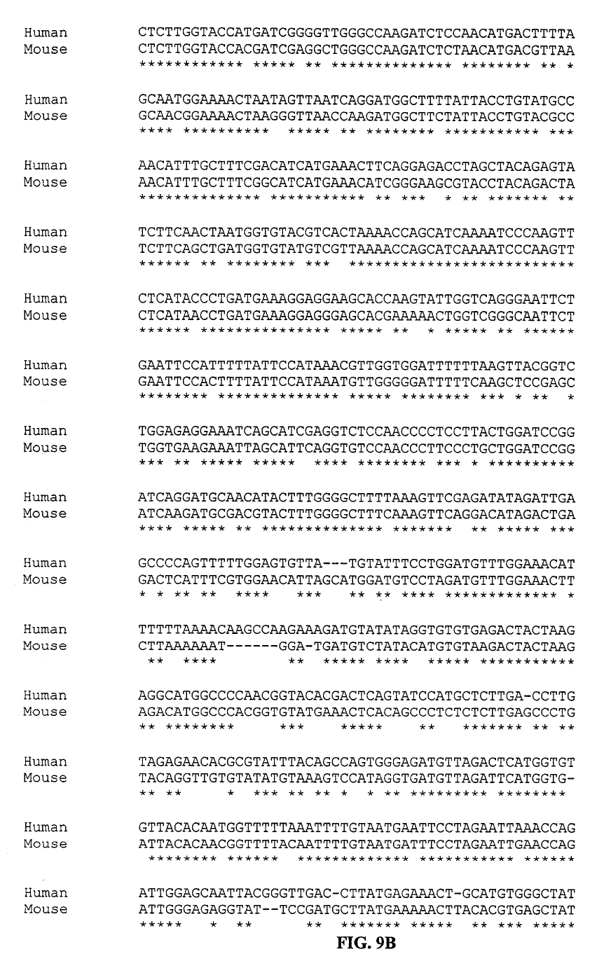

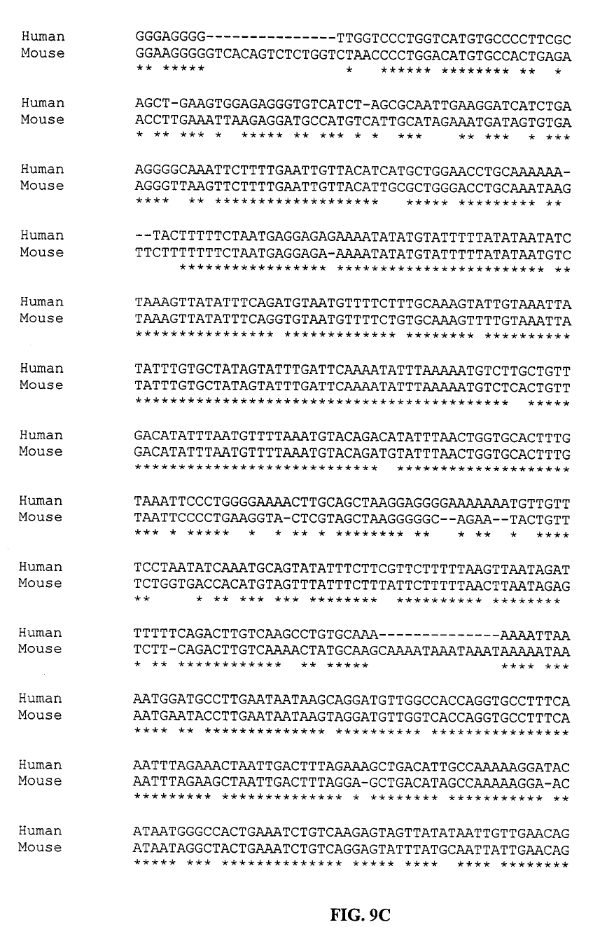

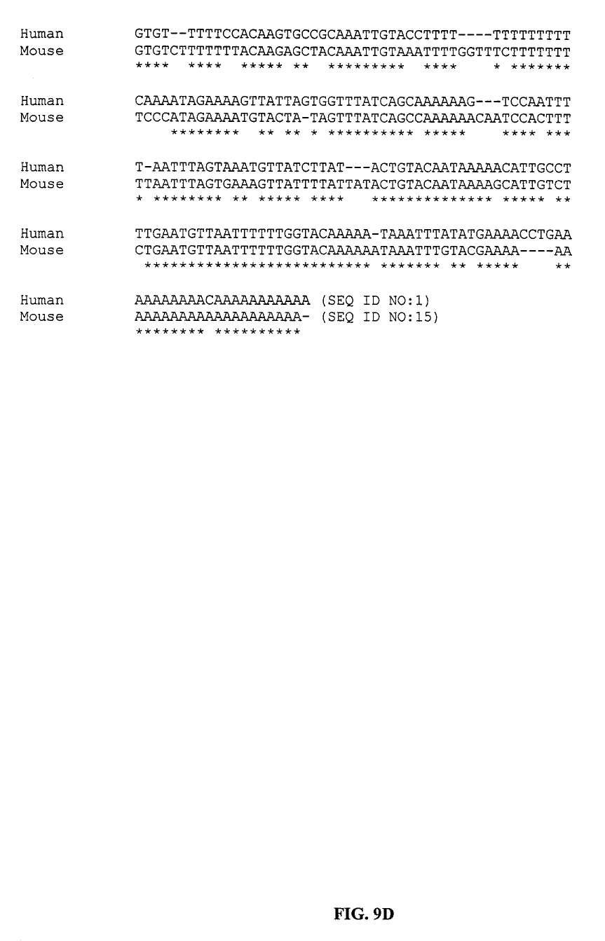

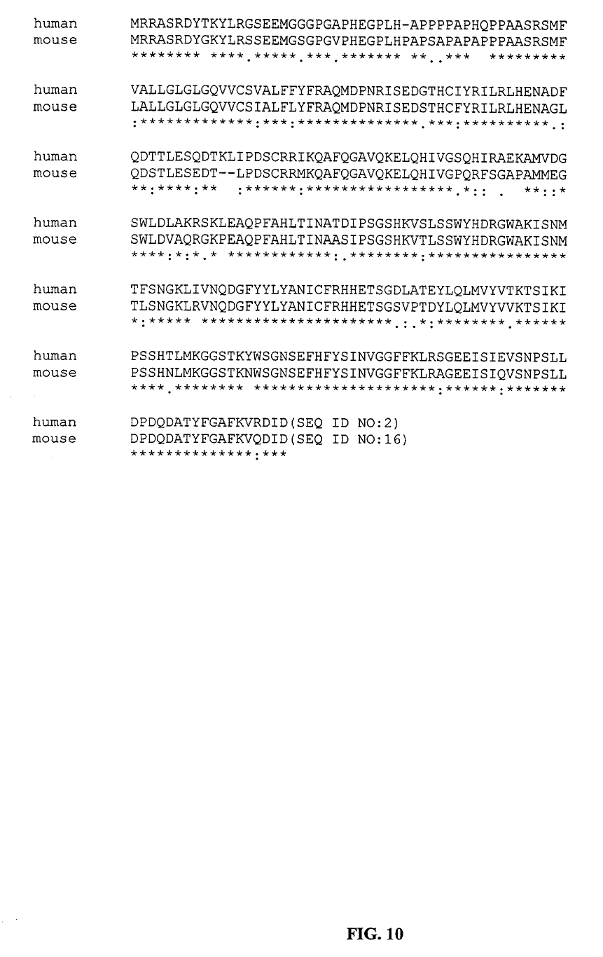

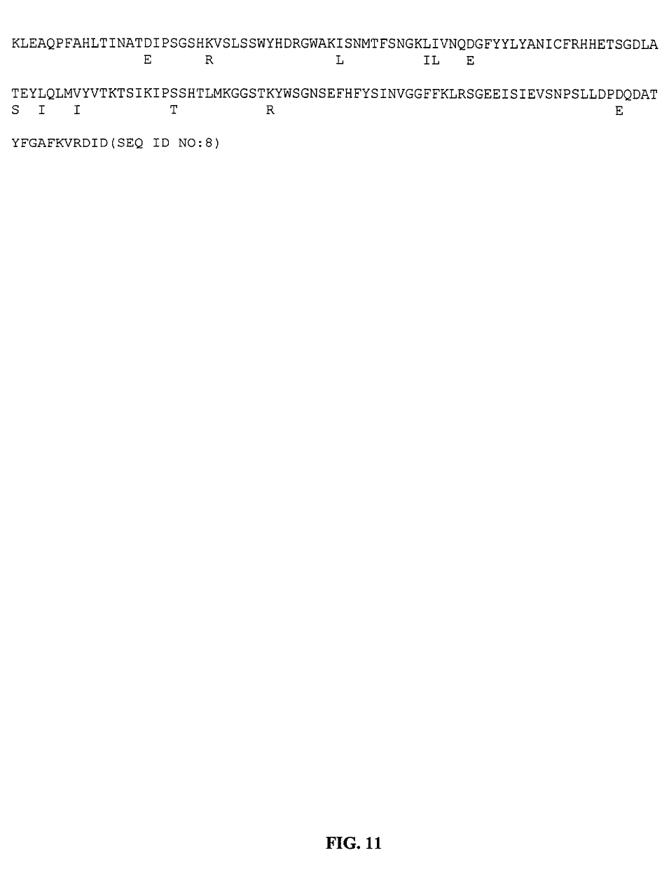

Some embodiments of the invention involve the use of a full-length TRANCE polypeptide or fragment thereof containing one or more mutations or modifications of a naturally-occurring TRANCE amino acid sequence. TRANCE polypeptides with altered amino acid sequences can be obtained by making random mutations in a naturally-occurring TRANCE sequence using random mutagenesis techniques known in the art. The resulting polypeptide is isolated and tested for a desired activity, activity as a TRANCE increasing or inhibiting agent, or increased in vivo half-life. Additionally, site-directed mutations can be engineered in the natural TRANCE amino acid sequence using conventional techniques. In designing TRANCE polypeptide variants, it is useful to distinguish between conserved positions and non-conserved positions in the TRANCE polypeptide. See, for example, a comparison of the mouse sequence with the human sequence in Lacey et al., 1998, Cell 93:165-176, and FIGS. 9A-D and 10. To produce variants with activity similar to that of the natural sequence, it is preferable not to alter conserved amino acid residues. It is also useful to distinguish conservative amino acid substitutions and non-conservative substitutions. Some embodiments of the invention involve the administration of a soluble, TNF domain-containing TRANCE polypeptide fragment containing one or more, e.g., 1-10, conservative amino acid substitutions relative to the corresponding segment of the naturally-occurring human TRANCE amino acid sequence (see, for example, FIG. 11). A “conservative amino acid substitution” is one in which an amino acid residue is replaced with another amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

A preferred method for producing TRANCE polypeptides for use in the invention is recombinant production, which involves genetic transformation of a host cell with a recombinant nucleic acid vector encoding a TRANCE polypeptide, expression of the recombinant nucleic acid in the transformed host cell, and collection and purification of the TRANCE polypeptide. Guidance concerning recombinant DNA technology can be found in numerous well-known references, including Sambrook et al., 1989, Molecular Cloning—A Laboratory Manual, Cold Spring Harbor Press; and Ausubel et al. (eds.), 1994, Current Protocols in Molecular Biology, John Wiley & Sons, Inc.

A recombinant, soluble, TNF domain-containing TRANCE polypeptide can be produced in a bacterial host such as E. coli. Such bacterial production can be direct expression of the desired polypeptide. Bacterial production also can be by expression of the desired TRANCE polypeptide fused to a bacterial signal sequence which directs secretion of the desired TRANCE polypeptide into the bacterial growth medium, with the signal sequence being removed in the secretion process. Alternatively, a recombinant, soluble, TNF domain-containing TRANCE polypeptide can be produced in a eukaryotic cell, e.g., a cultured human fibroblast cell. Expression of a full-length human TRANCE cDNA in cultured human fibroblasts results in the production of an approximately 45 kDa cell-associated TRANCE polypeptide and the release of an approximately 31 kDa soluble, C-terminal, TNF domain-containing TRANCE fragment into the fibroblast growth medium.

Purification of recombinant TRANCE polypeptides can be performed by conventional methods and is within ordinary skill in the art. The purification can include two or more steps, and one step can be affinity chromatography employing anti-TRANCE antibodies covalently linked to a solid phase chromatography support (beads) such as crosslinked agarose or polyacrylamide. Other useful purification steps include gel filtration chromatography and ion exchange chromatography.

TRAF6 Nucleic Acids and Polypeptides

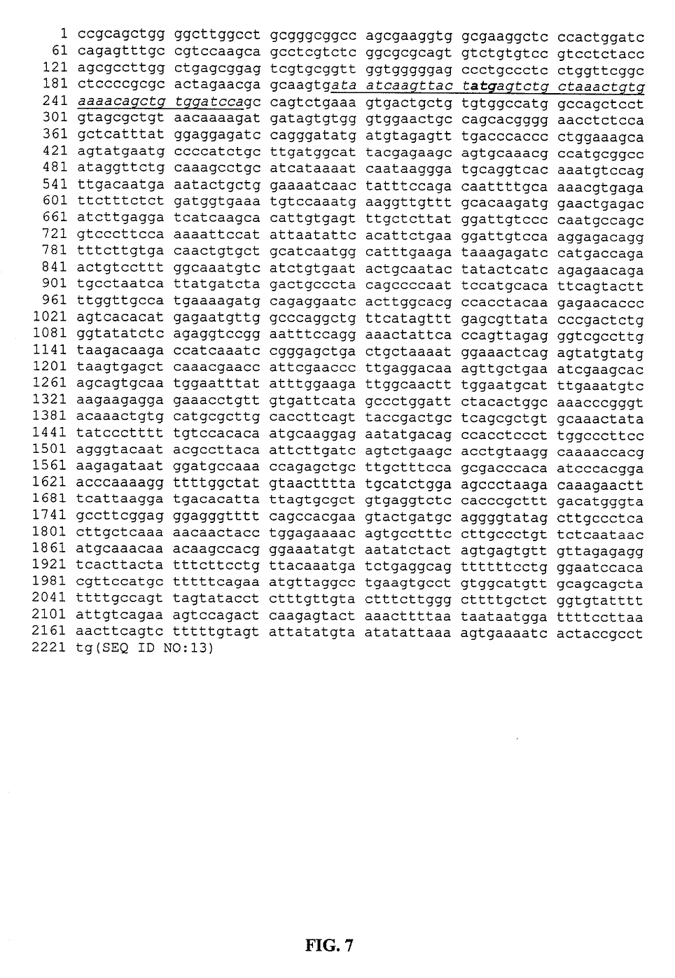

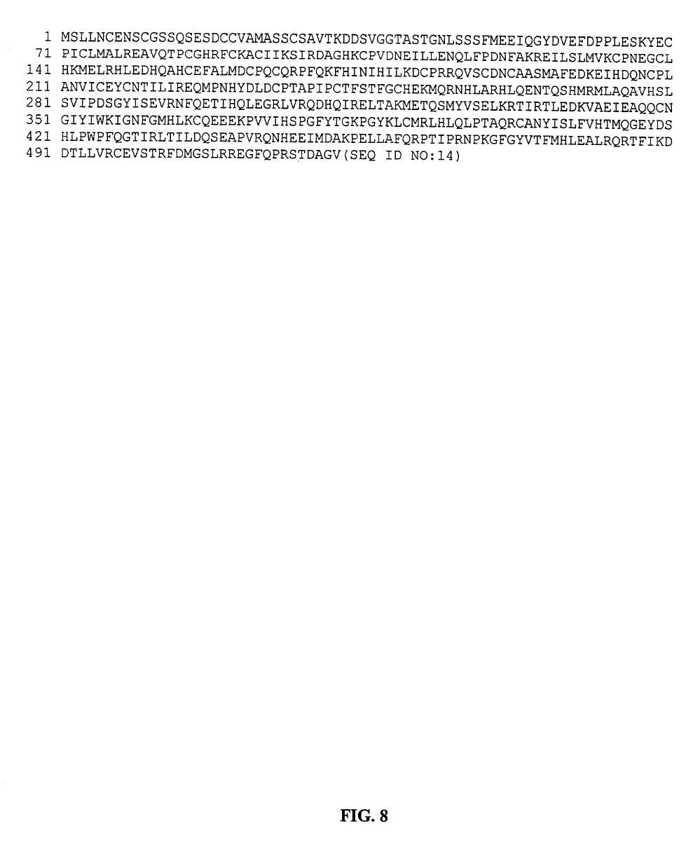

The complete amino acid sequence of a human TRAF6 polypeptide, the nucleotide sequence of a full-length cDNA encoding the human TRAF6 polypeptide, and methods for obtaining the TRAF6 DNA and polypeptide are found in Goeddel et al., U.S. Pat. No. 5,767,244. In addition, FIG. 7 depicts the Human TRAF6 cDNA, GenBank Accession # XM_{—}006284 (SEQ ID NO: 13). An example of a target region for antisense nucleic acids is shown italicized and underlined, with the translation start site in bold. FIG. 8 shows the Human TRAF6 amino acid sequence (SEQ ID NO: 14).

Pharmaceutical Compositions

The nucleic acid and polypeptides, fragments thereof, as well as antibodies, e.g., anti-TRANCE or anti-RANK antibody antibodies and other molecules and agents of the invention (also referred to herein as “active compounds”) can be incorporated into pharmaceutical compositions. Such compositions typically include the nucleic acid molecule, protein, or antibody and a pharmaceutically acceptable carrier. A “pharmaceutically acceptable carrier” can include solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifingal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be achieved by including an agent which delays absorption, e.g., aluminum monostearate and gelatin in the composition.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue, e.g., bone or cartilage, in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

For the TRANCE inhibiting or increasing agents described herein, an effective amount, e.g. of a protein or polypeptide (i.e., an effective dosage), ranges from about 0.001 to 30 mg/kg body weight, e.g. about 0.01 to 25 mg/kg body weight, e.g. about 0.1 to 20 mg/kg body weight. The protein or polypeptide can be administered one time per week for between about 1 to 10 weeks, e.g. between 2 to 8 weeks, about 3 to 7 weeks, or for about 4, 5, or 6 weeks. The skilled artisan will appreciate that certain factors influence the dosage and timing required to effectively treat a subject, including but not limited to the type of subject to be treated, the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

For antibodies, a useful dosage is 0.1 mg/kg of body weight (generally 10 mg/kg to 20 mg/kg). Generally, partially human antibodies and fully human antibodies have a longer half-life within the human body than other antibodies. Accordingly, lower dosages and less frequent administration are possible. Modifications such as lipidation can be used to stabilize antibodies and to enhance uptake and tissue penetration. A method for lipidation of antibodies is described by Cruikshank et al. ((1997) J. Acquired Immune Deficiency Syndromes and Human Retrovirology 14:193).

If the agent is a small molecule, exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

### EXAMPLES

The invention is further illustrated by the following examples. The examples are provided for illustrative purposes only, and are not to be construed as limiting the scope or content of the invention in any way.

Because of TRANCE's diverse effects in vivo, a knockout mouse was generated which was then “rescued” by a TRANCE transgene under the control of a lymphocyte-specific promoter. The skeletal manifestations in the knockout mouse and in the genetic “rescue” were then studied. The phenotypic effects of the transgene in the skeleton varied, depending on the tissue location. This indicated that normal skeletal growth and maintenance depend in large part upon a controlled, local supply of TRANCE.

### Example 1

### Tissue Preparation and Histology

Tibiae and femora were dissected and processed for histological and histochemical analyses as described in Seifert et al., 1988, Am. J. Anat. 183:158-165. Some bones were processed for thin sections by fixation in 2.5% glutaraldehyde in 0.1 M sodium cacodylate followed by decalcification in 10% disodium EDTA, after which they were dehydrated with ethanol and embedded in epoxy (Epon, Polysciences, Worthington, Pa.). Some tissues were processed for tartrate resistant acid phosphatase (TRAP) enzyme histochemistry with controls, by fixation in 2.5% glutaraldehyde in 0.1 M cacodylate supplemented with 7% glucose, dehydrated, and embedded in glycolmethacrylate. Brief treatment with 0.1% toluidine blue was used for histological staining. Some bones were processed without demineralization steps to assess the degree of mineralization in bones and cartilage.

### Example 2

### Radiological Impact of TRANCE Gene Deletion and Rescue

The overall skeletal impact of deleting the normal TRANCE gene, and then of “rescuing” the knockout (ko) with a lymphocyte-expressed transgene (tg/ko) was investigated. X-rays were taken at various ages of whole skeletons, of dissected limbs, and of skulls dissected at the midline and examined for skeletal phenotype, including the presence/absence of marrow spaces and erupted teeth (Cielinski et al., 1994, Arch. Oral Biol. 39:985-990; Cielinski et al, 1994, Bone 15:707-715). The thickness of epiphyseal growth plates and of residual sub-epiphyseal sclerosis in transgenic animals were assessed visually. Measurements of bone dimensions were made on radiographs projected at an enlargement of approximately 10× to permit greater precision. Tibial length was measured between the proximal and distal growth plates as described in Odgren et al., 1999, Bone 25:175-181. Tibial width was measured at the thickest part of the proximal epiphysis. Skull length was measured both from the posterior-most surface of the occipital bone to the posterior surface of the incisor (total length) and from the anterior edge of the tympanic bulla to the posterior surface of the incisor (anterior length). Standard statistical parameters and tests for significance of differences were obtained with Excel 97 (Microsoft, Redmond, Wash.).

The overall skeletal impact of deleting the normal TRANCE gene, and then of “rescuing” the knockout (ko) with a lymphocyte-expressed transgene (tg/ko) was evident in radiographs. The long bones of the hind limb, the vertebral column, the pelvis, and the skull were all shorter in the ko than in the wild type animals, the teeth did not erupt, and the ephiphyseal growth plates were abnormally thick. The transgene improved some, but not all, of the defects, suggesting the involvement of local delivery and/or developmental expression effects. Most notably, marrow spaces were restored in the appendicular and axial skeleton, and growth plate thickness appeared closer to normal. Strikingly, however, there remained dense, sclerotic regions of unresorbed mineralized cartilage at the ends of the marrow cavities, and the teeth did not erupt.

To assess these effects more quantitatively, radiographs were enlarged by projection, and a series of measurements was made on animals of the different genotypes at ages ranging up to three months. In ko animals, the tibia was only 68% of the normal length at 1 month and 65% at two months. The tg/ko animals showed slight improvement, being 70% and 77% of normal at two and three months, respectively. On the other hand, the width of the proximal tibia was not significantly different from normal in either the ko or the tg/ko animals at one month, but was greater than normal at two and three months (ko, 131% of normal at 2 months, P<0.001; tg/ko, 137% at 3 months, P<0.002). Skull lengths were also measured, both total length and the length from the anterior margin of the typanic bulla, to assess relative contributions of different centers of skull growth. Both measurements were affected similarly in the ko and tg/ko animals, being about 87% of the normal length at one month. The length of the same segment of the vertebral column (10 vertebrae moving caudally from the first sacral vertebra) in the different genotypes was also measured. The ko animals had significantly shorter spinal columns than wild type animals (77% at 1 month, P <0.0001; 74% at 2 months, P<0.01). The tg/ko animals showed improvement in this parameter, being 93% of normal by 3 months of age, not significantly different (P>0.1). These tests were also carried out comparing wild type mice and mice expressing the transgene in a wild type background. No significant differences were observed. In summary, the impact of the lymphocyte-expressed TRANCE transgene on growth varied depending on skeletal site, having no effect on either length or width of long bones or skull, while causing a substantial improvement in vertebral growth by 3 months of age. As previously mentioned, such variations suggest the involvement of developmental effects or the effects of local administration.

### Example 3

### Histological Impact of TRANCE Gene Deletion and Rescue

The histological impact of deleting the normal TRANCE gene, and then of “rescuing” the ko with a lymphocyte-expressed tg/ko was investigated. The following observations were made on demineralized 1 μm sections of proximal tibiae from animals at 1 month of age. The growth plates of both ko and tg/ko animals lacked the normal, columnar organization of chondrocytes, and had markedly reduced proliferating zones and increased hypertrophic zones, though the hypertrophic zones of tg/ko animals were less expanded. An orderly transition from bone to cartilage at the chondroosseous junction failed to occur in both ko and tg/ko animals. The mineralized cartilage and bone that filled the diaphysis in ko mice was histologically indistinguishable from the sclerotic tissue remaining at the metaphyseal ends of the tg/ko long bones, and was consistent with that material never having been resorbed or remodeled. These growth plate abnormalities provided a likely, histologically identifiable, cellular explanation for the growth defects observed in both the ko and tg/ko animals.

To examine in more detail the cellular basis for the skeletal phenotype of the ko and tg/ko mice, enzyme histochemistry was used to visualize tartrate-resistant acid phosphatase (TRAP), an osteoclast-specific marker. The proximal tibia of wild-type mice showed normal endochondral bone features, with the following key regions of osteoclast activity: (1) above the growth plate at the bottom of the epiphysis; (2) immediately below the growth plate at the chondroosseous junction; (3) along the trabeculae of the primary spongiosa; and (4) on the periosteal surface of the metaphysis, where the flared ends of the bone must be narrowed during growth. TRAP-positive cells were observed in all these areas in the wild-type mouse. No osteoclasts were seen in the TRANCE ko mouse. More than 75 sections from 8 individual TRANCE ko mice were observed, and no evidence of TRAP-positive cells was found in any of them.

The histology of the TRANCE tg/ko mice confirmed the radiologic evidence described above. A layer of dense, mineralized cartilage and bone was underling the growth plate, with an abrupt transition to a marrow space devoid of trabeculae. Osteoclasts were observed only in areas 1 and 3, i.e., above the growth plate and at the lower boundary of the sclerotic metaphysis. Examination of non-demineralized sections confirmed the presence of mineral in this region. Both the chondroosseous junction and the metaphyseal periosteum were devoid of osteoclasts in the tg/ko animals. A higher magnification histological section confirmed that the tg/ko mice were producing bona fide osteoclasts.

Osteoclasts appeared only on the endosteal surface of tg/ko long bones without involvement of periosteal osteoclasts. Thus, an investigation of whether the bone of the diaphysis undergoes normal remodeling was performed. In the tg/ko mice, normal, lamellar bone replaced the non-remodeled woven bone seen in the ko animals. It was noted that abundant osteocytes occupied lacunae within the layered extracellular matrix, with the lamellar organization of the bone evident from the endosteal surface through to the periosteal side. A high degree of variability in cortical thickness was observed in tg/ko mice at 2 and 3 months, ranging from normal in appearance to osteoporotic, and the thickness of the unresorbed sclerotic layer at the ends of long bones also varied. Preliminary evidence indicated that the degree of resorption activity correlates with individual variation in the number of TRANCE positive lymphocytes present in the marrow in tg/ko mice that is independent of gene dosage.

These results provided quantitative data on retarded skeletal growth at multiple sites in TRANCE knockout mice, confirmed that such mice have osteopetrosis and lack osteoclasts and erupted teeth, and demonstrated that a lymphocyte-mediated transgenic rescue has a positive effect on osteoclast differentiation and function in only a subset of their normal skeletal sites of action.

The growth retardation of endochondral bones in TRANCE ko and tg/ko mice correlated with a unique chondrodystrophy at the growth plates, i.e., a marked reduction of the proliferating chondrocyte zone and an increase in the zone of hypertrophy. This was in contrast to normal skeletal growth in which a remarkable constancy in the relative length of metaphyseal trabeculae is maintained at both ends of long bones as the central marrow cavity expands longitudinally. This is accomplished by a highly coordinated differentiation of chondrocytes in growth plates and the formation and resorption of bone in the metaphysis. Chondrocyte proliferation, hypertrophy, mineralization of vicinal matrix, and apoptosis in the orderly columns of growth plates is precisely balanced by vascular invasion of lacunae at the chondroosseous junction, removal of more than half of these columns at this site, deposition of bone onto the remaining mineralized cartilaginous scaffolds beginning at the chondroosseous junction, and removal of both mineralized cartilage and bone at the advancing marrow cavity ends of metaphyseal trabeculae.

Thus, in TRANCE knockout mice, growth is retarded, chondrocyte differentiation is deranged, and bone resorption is absent. The ability of a lymphocyte-delivered transgene to rescue marrow cavity appearance and expansion, but without an effect on resorption of the mineralized cartilaginous scaffold at the chondroosseous junction or on the chondrodystrophy, indicated that correcting a specific subset of the effects of the TRANCE-deficient state requires local delivery of the molecule.

### OTHER EMBODIMENTS

A number of embodiments of the invention have been described. Nevertheless, it will be understood that various modifications may be made without departing from the spirit and scope of the invention. For example, an antisense nucleic acid directed against a TRANCE mRNA and a TRANCE-binding agent can be administered in combination. Accordingly, other embodiments are within the scope of the following claims.

What is claimed is:

1. A method of treating a mammal having a disorder comprising insufficient cartilage growth or insufficient skeletal growth, the method comprising administering to the mammal an amount of a tumor necrosis factor-related activation induced cytokine (TRANCE)-inhibiting agent effective to increase cartilage growth or skeletal growth.

2. The method of claim 1, wherein the TRANCE-inhibiting agent is an antisense nucleic acid directed against a TRANCE RNA or a TRAF6 RNA.

3. The method of claim 2, wherein the antisense nucleic acid directed against a TRANCE RNA has the sequence of SEQ ID NO:17 or SEQ ID NO:18; and the antisense nucleic acid directed against the TRAF6 RNA has the sequence of SEQ ID NO: 19.

4. The method of claim 2, wherein the antisense nucleic acid is administered locally at a site of insufficient cartilage growth or insufficient skeletal growth in the mammal.

5. The method of claim 1, wherein the disorder is selected from the group consisting of dwarfism, osteopetrosis, craniofacial-skeletal discrepancies, and bone or cartilage damage resulting from traumatic injury, surgery, osteoarthritis or rheumatoid arthritis.

6. The method of claim 1, wherein the TRANCE-inhibiting agent is a TRANCE-binding molecule that sequesters TRANCE to form an inactive complex.

7. The method of claim 6, wherein the TRANCE-binding molecule is an anti-TRANCE antibody, or a TRANCE-binding fragment thereof.

8. The method of claim 6, wherein the TRANCE-binding molecule is an isolated RANK receptor, or a TRANCE-binding fragment thereof.

9. A method of treating a mammal having a disorder comprising excessive cartilage growth or excessive skeletal growth, the method comprising administering to the mammal an amount of a tumor necrosis factor-related activation induced cytokine (TRANCE)-increasing agent effective to decrease cartilage growth or skeletal growth.

10. The method of claim 9, wherein the TRANCE-increasing agent is a polypeptide comprising a tumor necrosis factor (TNF) domain of a TRANCE protein.

11. The method of claim 10, wherein the polypeptide is locally administered at a site of excessive cartilage growth or excessive skeletal growth in the mammal.

12. The method of claim 10, wherein the polypeptide comprises the amino acid sequence of SEQ ID NO:3, SEQ ID NO:4, SEQ ID NO:5, SEQ ID NO:6, SEQ ID NO:7, or SEQ ID NO:8, but lacks the cytoplasmic domain and transmembrane domain, of human TRANCE.

13. The method of claim 10, wherein the polypeptide consists of SEQ ID NO:3, SEQ ID NO:4, SEQ ID NO:5, SEQ ID NO:6, SEQ ID NO:7 or SEQ ID NO:8.

14. The method of claim 10, wherein the TRANCE protein is human TRANCE protein.

15. The method of claim 9, wherein the TRANCE-increasing agent is a TRAF6 polypeptide.

16. The method of claim 15, wherein the TRAF6 polypeptide is a human TRAF6 polypeptide.

17. The method of claim 15, wherein the method comprises introducing a TRAF6 polypeptide into a chondrocyte at a site of excessive cartilage growth or excessive skeletal growth in the mammal.

18. The method of claim 17, wherein introducing the TRAF6 polypeptide into the chondrocyte comprises locally administering an expression vector comprising a nucleotide sequence encoding a TRAF6 polypeptide operably linked to an expression control sequence, whereby the expression vector enters the chondrocyte and expresses the TRAF6 polypeptide in the chondrocyte.

19. The method of claim 15, wherein: (a) the TRAF6 polypeptide is linked to a membrane translocation moiety to form a cell-permeating TRAF6, and (b) the cell-permeating TRAF6 is locally administered at a site of insufficient cartilage growth or insufficient skeletal growth in the mammal.

20. The method of claim 9, wherein the disorder is selected from the group consisting of acromegaly, gigantism, exostosis cartilaginea, exostosis bursata, and multiple osteocartilaginous exostoses.

21. A method of promoting growth of cartilage in a mammal, the method comprising: removing cartilage from a mammal; culturing the cartilage in vitro; contacting chondrocytes in the cartilage with a tumor necrosis factor-related activation induced cytokine (TRANCE)-inhibiting agent; and reintroducing the cartilage into the mammal.

22. The method of claim 20, wherein the TRANCE-inhibiting agent is a TRANCE antisense nucleic acid.

23. The method of claim 20, wherein the TRANCE-inhibiting agent is a TRAF6 antisense nucleic acid.

24. The method of claim 20, wherein the TRANCE-inhibiting agent is a TRANCE-binding molecule.

25. A method of diagnosing a cartilage disorder in a mammal, the method comprising
obtaining a chondrocyte from the mammal;
detecting a level of tumor necrosis factor-related activation induced cytokine (TRANCE), receptor activator of NF-KB (RANK), or TNF-receptor-associated factor 6 (TRAF6) in the chondrocyte, wherein a level of TRANCE, RANK, or TRAF6 that is elevated or reduced compared to a normal level indicates the presence of a cartilage disorder in the mammal.

26. A method of identifying a candidate tumor necrosis factor-related activation induced cytokine (TRANCE)-inhibiting compound, the method comprising:
obtaining a cultured, proliferating test chondrocyte;
contacting the test chondrocyte with a test compound and a TRANCE polypeptide; and
detecting proliferation in the test chondrocyte compared to a control chondrocyte contacted with a TRANCE polypeptide unaccompanied by the test compound, whereby an increase in proliferation indicates that the test compound is a candidate TRANCE-inhibiting compound.

27. The method of claim 26, wherein the chondrocyte is selected from the group consisting of: a primary chondrocyte, a chondrocyte from a cultured chondrocyte cell line, a primary chondrocyte from a TRANCE null, transgenic non-human mammal, and a chondrocyte from a cultured chondrocyte cell line derived from a TRANCE null, transgenic non-human mammal.

28. A method of identifying a candidate tumor necrosis factor-related activation induced cytokine (TRANCE)-increasing compound, the method comprising:
obtaining a cultured, proliferating test chondrocyte;
contacting the test chondrocyte with a test compound and a TRANCE polypeptide; and
detecting proliferation in the test chondrocyte compared to a control chondrocyte contacted with a TRANCE polypeptide unaccompanied by the test compound, whereby a decrease in proliferation indicates that the test compound is a candidate TRANCE-increasing compound.

29. The method of claim 28, wherein the chondrocyte is selected from the group consisting of: a primary chondrocyte, a chondrocyte from a cultured chondrocyte cell line, a primary chondrocyte from a TRANCE null, transgenic non-human mammal, and a chondrocyte from a cultured chondrocyte cell line derived from a TRANCE null, transgenic non-human mammal.
